(19) United States
(12) Patent Application Publication
(10) Pub. No.: US 2025/0073497 A1
(43) Pub. Date: Mar. 6, 2025

(54) BIOMECHANICAL DATA AUGMENTATION FOR AI AND IMAGE-GUIDED RADIATION THERAPY

(71) Applicant: Siemens Healthineers International AG, Steinhausen (DE)

(72) Inventors: Anand SANTHANAM, Steinhausen (CH); Florin-Cristian GHESU, Steinhausen (CH); Yue ZHANG, Steinhausen (CH)

(21) Appl. No.: 18/239,068

(22) Filed: Aug. 28, 2023

(51) Int. Cl. A61N 5/10; G16H 20/40; G16H 40/40

(52) U.S. Cl. CPC A61N 5/1031; A61N 5/1067; G16H 20/40; G16H 40/40

(57) ABSTRACT

Disclosed herein are methods and systems for predicting how internal organs change and using the prediction in image-guided radiation therapy. A method comprises receiving an attribute of a radiation therapy treatment plan for a patient and at least one medical image of the patient; executing an artificial intelligence model to predict deformation data for at least one internal structure of the patient using the attribute and the at least one medical image, wherein the artificial intelligence model is trained in accordance with a training dataset comprising a set of participants and their corresponding deformation data; and outputting the predicted deformation data.

### TECHNICAL FIELD

This application relates generally to using data analysis techniques to model and predict patient attributes for radiation therapy treatment.

### BACKGROUND

One of the major challenges in image-guided radiation therapy (IGRT) is addressing various types of changes in patient conditions. One example of a change in patient condition is patient motion. The motion can be both cyclical motion (e.g., components of respiratory and cardiac motion) as well as irregular motion (e.g., gastrointestinal events including peristalsis, swallowing, the passage of gas bubbles, muscle relaxation in breath-hold, and body and limb movement). Another example may be a change in the patient's biometric attributes. For instance, as the patient continues treatment, the patient's tumor may exhibit various changes and deformations, such as overall volume shrinkage or deformations of the tumor, which is the ultimate goal of the patient's treatment. These volume changes may require revising the patient's radiation therapy treatment plan (RTTP).

### SUMMARY

For the aforementioned reasons, there is a desire for a system that can rapidly and accurately analyze patient information and provide a projected deformation of a patient's internal structures. Using the methods and systems discussed herein, a computer model (e.g., artificial intelligence (AI) model) can account for a patient's internal deformations and changes. Applications of these computer models can be in the field of real-time tissue tracking during radiation beam delivery, real-time motion visualization, retrospective and/or real-time delivered dose calculation, organ/segmentation (e.g., gross tumor volume (GTV)), specific dose tracking, outcome prediction, and image reconstruction.

Using the methods and systems discussed herein, an AI model may predict how a patient's internal structures, such as a planning target volume (PTV) and/or Organ at Risk (OAR) will deform due to the patient undergoing treatment. Moreover, the AI model discussed herein may be configured to generate a synthetic medical image that then can be ingested by other computer models for further analysis and/or be viewed by a treating physician.

Radiation therapy is a step in an often multi-faceted intervention for patients with inoperable cancers. Patients are prescribed a specific amount of radiation dose for their tumors and this dose delivery may be distributed for multiple days (or fractions). The treatment is generally planned by acquiring a simulation medical image, such as a Computer tomography (CT) image. The critical structures including the tumor will be carefully contoured to develop an RTTP that is suitable for the patient. However, during the therapy, anatomical changes in the patient's anatomy may occur thereby compensating the accuracy of the RTTP since the RTTP is closely associated with the patient's anatomy. A common variant in radiation therapy is image-guided radiotherapy (IGRT), where daily Cone Beam CT images (CBCT) of the patient's anatomy will be acquired for verification of the patient setup and the physiological regression before each treatment fraction. Since the tumors and the surrounding organs can deform, the patient positioning can also lead to an underdosing of the tumor and/or an overdosing of the critical organs.

Patients undergoing radiotherapy often lose significant body weight and undergo tumor shrinkage. These factors lead to changes in the patient's anatomy that are referred to as a “physiological regression” and are considered systematic in nature. Accordingly, such regression may cause the RTTP to underdose or overdose the tumor/PTV. Moreover, the regression may also cause the RTTP to overdose critical organs that surround the tumor (OARs). Such physiological regression would require the treatment to be adapted as and when needed.

Using the methods and systems discussed herein, an AI model can be trained to predict the deformations and regressions at different times for the patients, such that the patient's RTTP can be dynamically adjusted accordingly.

Some conventional methods use existing and static algorithms to achieve results. However, using these algorithms and registries does not always result in efficient or accurate results. Tracking both patient setup and physiological regression requires the ability to deformably register the simulation CT anatomy with periodic (e.g., daily) imaging (CBCT). The monitored data may then be analyzed using algorithms commonly known as deformable image registration (DIR). However, using the DIR alone is not desirable. First, continuously monitoring a patient is a time-consuming endeavor that requires a high number of imaging to be performed.

Moreover, the DIR process is complicated by the quality of the daily imaging, normal physiological changes (e.g., bladder and rectal filling, lung volume changes due to treatment), patient physiological regression due to the therapy, and patient setup errors. While the quality of the daily imaging remains the same for a given treatment equipment, the remaining source of complexity varies from patient to patient. The DIR accuracy can depend on the DIR's hyper-parameters. Variations in the physiological regression and any setup errors may compensate for the DIR accuracy as it may require a different set of DIR hyper-parameters. This makes the task of achieving optimal DIR accuracy a circular problem. As used herein, a DIR hyperparameter may refer to tunable parameters that control the behavior and performance of the registration algorithm used in association with the DIR. These parameters influence various aspects of the registration process, such as the smoothness of the deformation field, the convergence criteria, and the regularization of the transformation.

Organ deformations and physiological regression may be systematic in nature and so can be represented by building an accurate AI model that analyzes bio-mechanical aspects of a patient's structures. Accordingly, the AI models discussed herein may be a bio-mechanical model in nature. The AI model can also act as a “digital twin” in enabling the prediction of different changes that may happen to the patient's internal structures during the course of treatments. The AI model can be actuated in a specific manner to generate the deformations associated with a specific regression pattern. The resulting deformations can be used to optimize and improve the DIR accuracy so that the analytics server can then predict the DIR hyper-parameters.

Using the digital twin approach may bring together time series processing and computer vision by introducing a convolutional recurrent cell in each layer of the AI model. In this approach, the analytics server/AI model may formulate the prediction problem as a spatiotemporal sequence forecasting problem that can be solved under the general sequence-to-sequence learning framework. In order to model the spatiotemporal relationships between the anatomical structures, the analytics server may employ the twin AI model as a transformer, which has convolutional structures in both the input-to-state and state-to-state transitions. By stacking multiple layers and forming a transformer structure, the analytics server may assemble an end-to-end trainable AI model for predicting the DIR hyperparameters changes.

Accordingly, using the methods and systems discussed herein a server can train optimizers for improving DIR accuracy using biomechanical models as a surrogate for actual patient changes. One of the advantages of this approach stems from the fact that given the pair of simulation CT and/or CBCT, it will be possible to obtain the optimal DIR performance that will compute the patient's anatomical changes and will eventually lead to computing the treatment adaptation (e.g., RTTP changes). The methodology may not be impacted by the random errors in the patient positioning and may also ensure that the tumor underdosing and organ overdosing can be predicted for the upcoming fractions and avoided. The approach presented herein may also be agnostic to the type of radiation therapy treatment equipment. Moreover, the approach presented herein may be agnostic to certain patient biometric attributes. For instance, the same approach may be applied to different patients having different BMIs.

In an embodiment, a method comprises receiving, by a processor, an attribute of a radiation therapy treatment plan for a patient and at least one medical image of the patient; executing, by the processor, an artificial intelligence model to predict deformation data for at least one internal structure of the patient using the attribute and the at least one medical image, wherein the artificial intelligence model is trained in accordance with a training dataset comprising a set of participants and their corresponding deformation data; and outputting, by the processor, the predicted deformation data.

The deformation data may correspond to a movement, volume expansion, or volume shrinkage of at least one internal structure of the patient.

The deformation data may be a hyperparameter used by a second model configured to predict a movement, volume expansion, or volume shrinkage of at least one internal structure of the patient.

The method may further comprise transmitting, by the processor, the hyperparameter to the second model.

The method may further comprise transmitting, by the processor, the deformation data to a plan optimizer computer model.

The method may further comprise adjusting, by the processor, at least one attribute of a radiation therapy machine in accordance with the predicted deformation data.

The deformation data may correspond to one or more deformation vectors.

In another embodiment, a non-transitory machine-readable storage medium having computer-executable instructions stored thereon that, when executed by one or more processors, cause the one or more processors to perform operations comprising receive an attribute of a radiation therapy treatment plan for a patient and at least one medical image of the patient; execute an artificial intelligence model to predict deformation data for at least one internal structure of the patient using the attribute and the at least one medical image, wherein the artificial intelligence model is trained in accordance with a training dataset comprising a set of participants and their corresponding deformation data; and output the predicted deformation data.

The deformation data may correspond to a movement, volume expansion, or volume shrinkage of at least one internal structure of the patient.

The deformation data may be a hyperparameter used by a second model configured to predict a movement, volume expansion, or volume shrinkage of at least one internal structure of the patient.

The instructions may further cause the processor to transmit the hyperparameter to the second model.

The instructions may further cause the processor to transmit the deformation data to a plan optimizer computer model.

The instructions may further cause the processor to adjust at least one attribute of a radiation therapy machine in accordance with the predicted deformation data.

The deformation data may correspond to one or more deformation vectors.

In another embodiment, a system comprising a processor configured to receive an attribute of a radiation therapy treatment plan for a patient and at least one medical image of the patient; execute an artificial intelligence model to predict deformation data for at least one internal structure of the patient using the attribute and the at least one medical image, wherein the artificial intelligence model is trained in accordance with a training dataset comprising a set of participants and their corresponding deformation data; and output the predicted deformation data.

The deformation data may correspond to a movement, volume expansion, or volume shrinkage of at least one internal structure of the patient.

The deformation data may be a hyperparameter used by a second model configured to predict a movement, volume expansion, or volume shrinkage of at least one internal structure of the patient.

The instructions may further cause the processor to transmit the hyperparameter to the second model.

The instructions may further cause the processor to transmit the deformation data to a plan optimizer computer model.

The instructions may further cause the processor to adjust at least one attribute of a radiation therapy machine in accordance with the predicted deformation data.

In another embodiment, a method comprises a method comprises receiving, by a processor, an attribute of a radiation therapy treatment plan for a patient and at least one medical image of the patient; executing, by the processor, an artificial intelligence model to predict deformation data for at least one internal structure of the patient using the attribute and the at least one medical image, wherein the artificial intelligence model is trained in accordance with a training dataset comprising a set of participants and their corresponding deformation data; and generating, by the processor using the artificial intelligence model, a synthetic medical image representing the at least one medical image deformed in accordance with the predicted deformation data.

The synthetic medical image may be a different type than the at least one medical image.

The synthetic medical image is one of a computer tomography image, a cone beam computer tomography image, or a magnetic resonance image.

The deformation data may correspond to a movement, volume expansion, or volume shrinkage of at least one internal structure of the patient, the method further comprising transmitting, by the processor, the synthetic medical image to a second model.

The method may further comprise transmitting, by the processor, the deformation data to a plan optimizer computer model.

The method may further comprise adjusting, by the processor, at least one attribute of a radiation therapy machine in accordance with the predicted deformation data.

The synthetic medical image may comprise a deformation vector indicating a movement of at least one segment of the patient's internal structure.

In another embodiment, a non-transitory machine-readable storage medium has computer-executable instructions stored thereon that, when executed by one or more processors, cause the one or more processors to perform operations comprising receive an attribute of a radiation therapy treatment plan for a patient and at least one medical image of the patient; execute an artificial intelligence model to predict deformation data for at least one internal structure of the patient using the attribute and the at least one medical image, wherein the artificial intelligence model is trained in accordance with a training dataset comprising a set of participants and their corresponding deformation data; and generate, using the artificial intelligence model, a synthetic medical image representing the at least one medical image deformed in accordance with the predicted deformation data.

The synthetic medical image may be a different type than the at least one medical image.

The medical image may be one of a computer tomography image, a cone beam computer tomography image, or a magnetic resonance image.

The deformation data may correspond to a movement, volume expansion, or volume shrinkage of at least one internal structure of the patient, wherein the instructions further cause the processor to transmit the synthetic medical image to a second model.

The instructions may further cause the processor to transmit the deformation data to a plan optimizer computer model.

The instructions may further cause the processor to adjust at least one attribute of a radiation therapy machine in accordance with the predicted deformation data.

The synthetic medical image may comprise a deformation vector indicating a movement of at least one segment of the patient's internal structure.

In another embodiment, a system comprising a processor configured to receive an attribute of a radiation therapy treatment plan for a patient and at least one medical image of the patient; execute an artificial intelligence model to predict deformation data for at least one internal structure of the patient using the attribute and the at least one medical image, wherein the artificial intelligence model is trained in accordance with a training dataset comprising a set of participants and their corresponding deformation data; and generate, using the artificial intelligence model, a synthetic medical image representing the at least one medical image deformed in accordance with the predicted deformation data.

The synthetic medical image may be a different type than the at least one medical image.

The medical image may be one of a computer tomography image, a cone beam computer tomography image, or a magnetic resonance image.

The deformation data may correspond to a movement, volume expansion, or volume shrinkage of at least one internal structure of the patient, wherein the instructions further cause the processor to transmit the synthetic medical image to a second model.

The processor may be further configured to transmit the deformation data to a plan optimizer computer model.

The processor may be further configured to adjust at least one attribute of a radiation therapy machine in accordance with the predicted deformation data.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of an AI-based bio-mechanical modeling system, according to an embodiment.

FIG. 2 illustrates a process flow diagram of an AI-based bio-mechanical modeling system, according to an embodiment.

FIG. 3 illustrates a process flow diagram of an AI-based bio-mechanical modeling system, according to an embodiment.

FIG. 4 illustrates a visual representation of deformation vectors, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

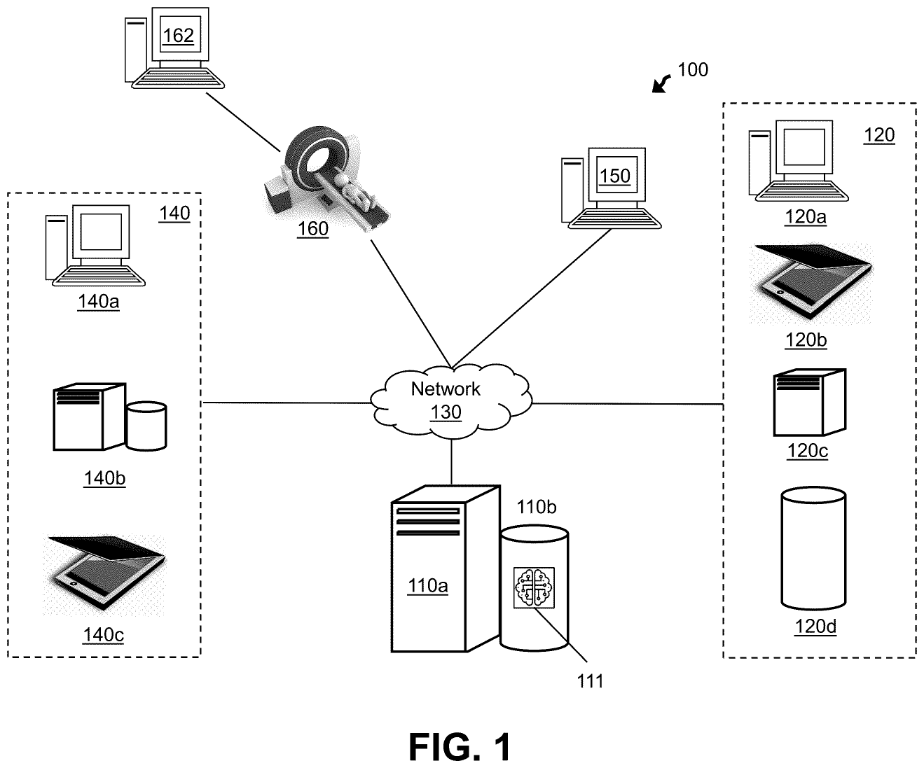

FIG. 1 illustrates components of a system 100 for an artificial intelligence bio-mechanical modeling system, according to an embodiment. The system 100 may include an analytics server 110a, system database 110b, an AI model 111, electronic data sources 120a-d (collectively electronic data sources 120), end-user devices 140a-c (collectively end-user devices 140), an administrator computing device 150, and medical device 160, medical device computer(s) 162. Various components depicted in FIG. 1 may belong to a radiation therapy clinic at which patients may receive radiation therapy treatment, in some cases via one or more radiation therapy machines located within the clinic (e.g., medical device 160).

The system 100 is not confined to the components described herein and may include additional or other components, not shown for brevity, which is to be considered within the scope of the embodiments described herein.

The above-mentioned components may be connected to each other through a network 130. Examples of the network 130 may include, but are not limited to, private or public local-area-networks (LAN), wireless LAN (WLAN) networks, metropolitan area networks (MAN), wide-area networks (WAN), and the Internet. The network 130 may include wired and/or wireless communications according to one or more standards and/or via one or more transport mediums. The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, e.g., a GSM (Global System for Mobile Communications), CDMA (Code Division Multiple Access), and EDGE (Enhanced Data for Global Evolution) network.

The analytics server 110a may generate and display an electronic platform configured to use various AI model 111 (including artificial intelligence and/or machine learning models) for receiving patient information and outputting the results of execution of the AI model 111. The electronic platform may include graphical user interfaces (GUI) displayed on each electronic data source 120, the end-user devices 140, the medical device 160, and/or the administrator computing device 150. An example of the electronic platform generated and hosted by the analytics server 110a may be a web-based application or a website configured to be displayed on different electronic devices, such as mobile devices, tablets, personal computers, and the like.

The information displayed by the electronic platform can include, for example, input elements to receive data associated with a patient being treated and display results of predictions produced by the AI model 111 (e.g., a synthetic medical image for the patient that displays a projected location of a tumor predicted by the AI 111). For instance, the analytics server 110a may execute the AI model 111 to generate predicted tumor attributes for a patient being treated via the medical device 160, such as tumor location, deformation data, and the like. The analytics server 110a may then display the results for a medical professional and/or directly revise one or more operational attributes of the medical device 160. In some embodiments, the medical device 160 can be a diagnostic imaging devices or a treatment delivery device.

The analytics server 110a may be any computing device comprising a processor and non-transitory machine-readable storage capable of executing the various tasks and processes described herein. The analytics server 110a may employ various processors such as central processing units (CPU) and graphics processing units (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The electronic data sources 120 may represent various electronic data sources that contain, retrieve, and/or access data needed to train and execute the AI model 111. In some embodiments, the electronic data sources may include data associated with operational and treatment information associated with previously performed radiation therapy treatments, data associated with previously monitored patients (e.g., previous medical images illustrating treated patient's internal structures), or participants in a study to train the AI models discussed herein. For instance, the analytics server 110a may use the clinic computer 120a, medical professional device 120b, server 120c (associated with a physician and/or clinic), and database 120d (associated with the physician and/or the clinic) to retrieve/receive data associated with patients, their treatment, and their medical images. In some embodiments, the electronic data sources 120 may include a server or processor associated with various DIR algorithms and processes. As discussed herein, the AI model 111 may use DIR data, in conjunction with other data, to achieve the results discussed herein, such as predicting the deformation and internal structure of the patient.

The analytics server 110a may retrieve the data from the end-user devices 120, generate a training dataset, and train the AI models 111. If needed, the analytics server 110a may execute various algorithms to translate raw data received/retrieved from the electronic data sources 120 into machine-readable objects that can be stored and processed by other analytical processes as described herein.

End-user devices 140 may be any computing device comprising a processor and a non-transitory machine-readable storage medium capable of performing the various tasks and processes described herein. Non-limiting examples of an end-user device 140 may be a workstation computer, laptop computer, tablet computer, and server computer. In operation, various users may use end-user devices 140 to access the GUI operationally managed by the analytics server 110a. Specifically, the end-user devices 140 may include clinic computer 140a, clinic server 140b, and a medical professional device 140c. Even though referred to herein as “end-user” devices, these devices may not always be operated by end-users. For instance, the clinic server 140b may not be directly used by an end user. However, the results stored onto the clinic server 140b may be used to populate various GUIs accessed by an end user via the medical professional device 140c. In a non-limiting example, the analytics server 110a may display a synthetic medical image on a platform presented on an end-user device 140.

The administrator computing device 150 may represent a computing device operated by a system administrator. The administrator computing device 150 may be configured to display radiation therapy treatment attributes generated by the analytics server 110a (e.g., various analytic metrics determined during training of one or more machine learning models and/or systems); monitor various models (e.g., AI model 111) utilized by the analytics server 110a, electronic data sources 120, and/or end-user devices 140; review feedback; and/or facilitate training or retraining (calibration) of the AI model 111 that are maintained by the analytics server 110a.

The medical device 160 may be a radiation therapy machine configured to implement a patient's radiation therapy treatment. The medical device 160 may also include an imaging device capable of emitting radiation such that the medical device 160 may perform imaging according to various methods to accurately image the internal structure of a patient. For instance, the medical device 160 may include a rotating system (e.g., a static or rotating multi-view system). A non-limiting example of a multi-view system may include stereo systems (e.g., two systems may be arranged orthogonally). The medical device 160 may also be in communication with a medical device computer 162 that is configured to display various GUIs discussed herein. For instance, the analytics server 110a may display the results predicted by the AI model 111 onto the computing devices described herein.

The AI model 111 may be stored in the system database 110b. The AI model 111 may be trained using data received/retrieved from the electronic data sources 120 and may be executed using data received from the end-user devices, and/or the medical device 160. In some embodiments, the AI model 111 may reside within a data repository local or specific to a clinic. In various embodiments, the AI models 111 use one or more deep learning engines to generate predicted organ deformity for a patient being treated.

It should be understood that any alternative and/or additional machine learning model(s) may be used to implement similar learning engines. The deep learning engines can include processing pathways that are trained during a training phase. Once trained, deep learning engines may be executed (e.g., by the analytics server 110a) to generate predicted patient attributes.

As described herein, the analytics server 110a may store the AI model 111 (e.g., neural networks, random forest, support vector machines, regression models, recurrent models, etc.) in an accessible data repository. The analytics server 110a may retrieve the AI model 111 and train the AI model 111 to predict a deformity associated with one or more of the patient's structures/organs.

Various machine learning techniques may involve “training” the machine learning models to predict (e.g., estimate the likelihood of) patient attributes, including supervised learning techniques, unsupervised learning techniques, or semi-supervised learning techniques, among others. In a non-limiting example, the predicted patient attribute may indicate a patient's predicted tumor attributes (e.g., location, volume change, deformation data) or deformity of the patient's other structures (e.g., OARs). The AI model 111 can therefore be used to predict the real-time location and orientation of the PTV. As a result, the analytics server 110a may display the tumor's projected location and/or revise the patient's treatment accordingly, such as by changing the MLC openings.

In practice, training data may be user-generated through observations and experience to facilitate supervised learning. For example, training data may be received and monitored during past radiation therapy treatments provided to prior patients. In another example, the training data may be a dataset that includes tumor deformation data of patients while undergoing treatment (e.g., losing weight and shrinking the tumor) and their corresponding movements (e.g., corresponding timestamped medical images). Training data may be pre-processed via any suitable data augmentation approach (e.g., normalization, encoding, any combination thereof, etc.) to produce a new dataset with modified properties to improve model generalization using ground truth.

The methods and systems described herein are not limited to training AI models based on patients who have been previously treated. For instance, instead of previously treated patients, the training dataset may include data associated with any set of participants (not patients) who are willing to be monitored for the purposes of generating the training dataset.

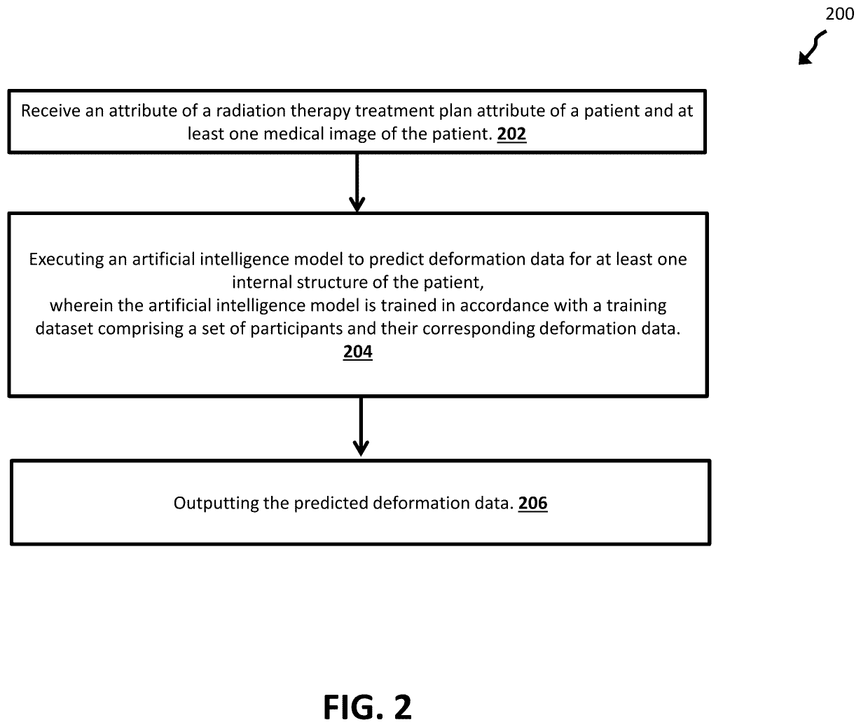

FIG. 2 depicts an example data flow diagram 200 that shows how an AI model can be trained and executed to predict a patient attribute (e.g., deformation data), in accordance with an embodiment. The method 200 may include steps 202-206. However, other embodiments may include additional or alternative steps or may omit one or more steps altogether. The method 200 is described as being executed by a server, such as the analytics server described in FIG. 1. However, one or more steps of method 200 may be executed by any number of computing devices operating in the distributed computing system described in FIG. 1. For instance, one or more computing devices may locally perform part or all of the steps described in FIG. 2.

Using the method 200, an AI model can be trained in accordance with a training dataset to predict how a patient's internal structures would change (e.g., move, expand, shrink, or deform) and consequently how the patient's deformation data affects the patient's RTTP attributes. At implementation time, using the method 200, the AI model may be executed to predict how a patient's internal structures are changing based on the patient's medical images and RTTP. As used herein, the deformation of an internal structure may refer to any volumetric expansion, volumetric reduction/shrinkage, and/or any movement of the internal structure.

### Training of the AI Model

Before executing the AI model, the analytics server may first train the AI model and ensure its accuracy. When trained, the AI model can provide a biomechanically guided prediction of the DTR hyperparameters that may provide the optimal DIR results between the simulation CT and the daily imaging and may ultimately lead to a treatment adaptation.

The analytics server may train the AI model using a training dataset comprising two sets of data associated with a cohort of patients (or participants in a clinical trial). First, the training dataset may include treatment data and other patient information associated with each patient. For instance, the training dataset may include all the data associated with the patient that is available, such as diagnosis, tumor location, patient's anatomy data, and the like. Second, the training dataset may include structure deformation data associated with the set of patients. For instance, the training dataset may include medical images associated with the set of patients. The medical images may be periodically obtained while the patients are undergoing treatment. Each image may be taken from a particular anatomical region of the patient. For instance, in operation and in order to prepare a training dataset, medical images of patients and participants are periodically taken. Each image may include a timestamp that can be used to identify corresponding treatment/patient data associated with the medical image.

In an embodiment, the training dataset may include medical images (e.g., CT, 4DCT, or MR) depicting the patient's internal organs. In some embodiments, the training dataset may also include MR and/or time-resolved MR data associated with the patient.

The training dataset may also include additional data associated with the patients. For instance, the AI model may consider each patient's demographic information and/or other biological markers (e.g., age, weight, or BMI). As a result, the model may also consider the patient's attributes when considering and relating how the patient's internal structures are deforming/moving. In operation, some patients may lose weight during (and as a result of) the treatment. Therefore, they may exhibit different deformation data.

When reviewed in totality, the training dataset may include information that could indicate how each patient's treatment affects their internal organs and structures. Specifically, the training dataset may indicate how undergoing treatment deforms or moves one or more internal structures of each patient. The analytics server may then aggregate various datasets that are associated with the set of patients and include the aggregated datasets within the training dataset. Using the training dataset, the analytics server may train one or more AI models discussed herein.

The AI model may analyze the images (including how the internal structures are moving) and their corresponding treatment/patient data to train itself, such that the trained AI model can ingest an initial medical image (or other patient data) associated with a new patient and predict how the new patient's internal structures would move and deform. The AI model may use a variety of methods to uncover hidden patterns, such as using deep learning methods. The AI model may use an unsupervised or semi-supervised method in which moving images are automatically analyzed and deformations are highlighted.

In some embodiments, the AI model may compare medical images for the patient and identify how each voxel has moved/deformed. To achieve this, the AI model may compute a difference between the images and test its identification of the difference by comparing pixel intensity values (e.g., Hounsfield unit (HU) for CT/CBCT) between the different medical images. Specifically, each image may be divided into different segments (e.g., pixels or a collection of pixels) and pixel intensity values of corresponding segments may be compared to determine how internal structures have moved.

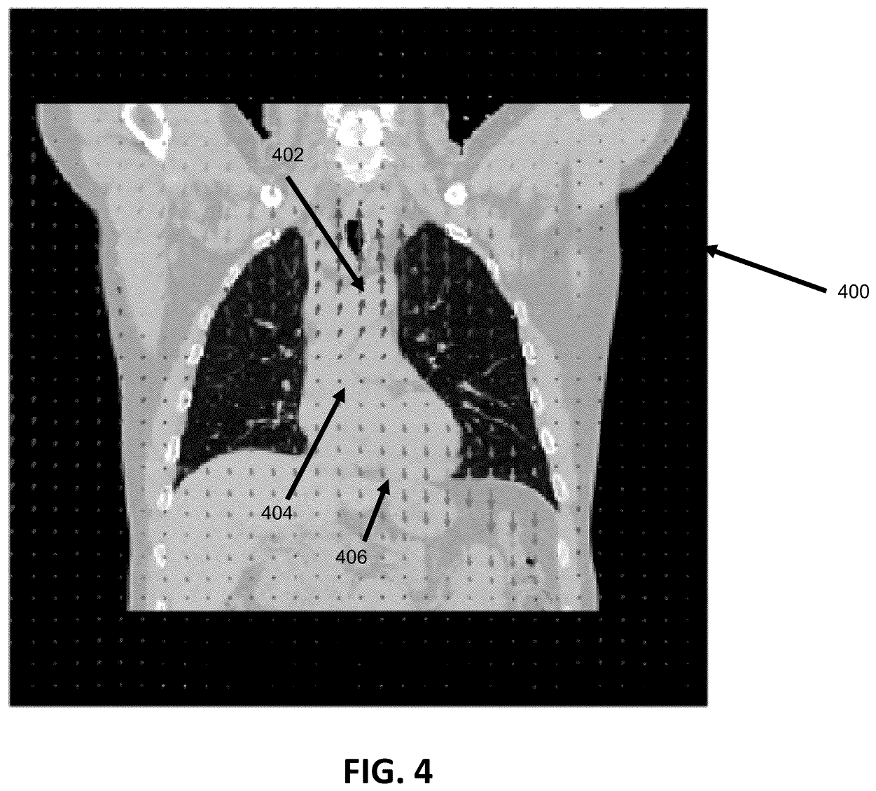

Referring now to FIG. 4, a non-limiting example of identifying movement using a medical image is illustrated. The AI model may retrieve two or more sets of medical images of the patient undergoing treatment and compare different voxels of the PTV and/or OARs. Specifically, using various methods (e.g., HU comparison), the AI model may determine a deformation vector for different structures of the patient, as depicted in FIG. 4. For instance, the medical image 400 illustrates a combination of two medical images and a variety of vectors that each indicate a movement (or sometimes non-movement) of a segment of the patient structures depicted. The AI model may consider any segment of the patient and then compare the same segments in the two or more medical images. Segments can be any number of pixels within the medical image. In some embodiments, to be more efficient, the AI model may combine multiple pixels and analyze them, as opposed to evaluating every pixel individually.

After comparing the medical images, the AI model may compute a deformation vector for different segments. The deformation vectors may indicate how each segment within a medical image of the patient will move/deform. The deformation vectors may indicate the distance and direction in which each segment within the medical image will move. For instance, as depicted, a vector 402 indicates that its corresponding segment within the medical image will move upwards (e.g., by 1 millimeter) and a vector 404 indicates that its corresponding location will not move. In contrast, a vector 406 indicates that its corresponding location will move downwards (e.g., 0.5 millimeters). Using the deformation vectors, the analytics server may train itself to predict the location and orientation of one or more internal structures of the patient.

The AI model may also be trained to generate a predicted deformation medical image for the patient. For instance, the AI model can be used to generate a moving or fixed medical image representing how the patient's internal structures will move/deform. The AI model may be trained to use one or more medical images of the patient and apply various predicted deformation vectors to one or more segments of the patient's actual medical image to generate a predicted (and sometimes referred to as “synthetic”) medical image.

Using various generative AI techniques, the AI model may simulate a medical image to convey the predicted deformation data of a patient. The AI model may leverage deep learning techniques to generate synthetic medical images. In some embodiments, the AI model may use generative adversarial networks (GANs) and variational autoencoders (VAEs) to learn complex representations and statistical patterns from extensive medical imaging datasets. The AI model may train itself by capturing and modeling the underlying distributions of pixel intensities, anatomical structures, and pathological features, such that the AI model can generate synthetic medical images that closely resemble real-world scans.

In some embodiments, the AI model's generation of the synthetic medical image may involve transforming the medical images to a latent space (e.g., a low-dimensional representation of the medical image) and encoding key features and variations within the medical images. The AI model may then manipulate various attributes in the latent space, such as applying predicted deformation vectors to anatomical shapes and/or imaging parameters. Using these methods, the AI model can generate a synthetic medical images that exhibits characteristics of the same internal structure of the patient deformed in accordance with the predicted deformation vectors.

In some configurations, the analytics server may pre-train or partially train the AI model. For instance, the analytics server may train the AI model based on a set of cohort patients. Then, the analytics server may train (fine-tune) the AI model using a particular patient's specific data. For instance, when the AI model is pre-trained, the analytics server may fine-tune the AI model and customize it to a particular patient by feeding information (e.g., diagnoses data, biometric data, and/or medical images) of the patient.

### Execution and Implementation of the AI Model

After the AI model is trained, the AI model can predict deformation vectors for a new patient. The deformation vectors identify how each segment within an image will move/deform.

Referring back to FIG. 2, at step 202, the analytics server may receive at least one radiation therapy treatment plan attribute of a patient and at least one medical image of the patient. The analytics server may receive patient data from one or more electronic data sources, such as by querying and retrieving the information or by receiving input from one or more clinicians and/or treating physicians. The data received may include the patient's RTTP (at least one attribute) and/or a medical image of the patient that illustrates the patient's internal organs, such as the PTV and OARs.

In a non-limiting example, during the planning process (when the analytics server is attempting to generate an RTTP for the patient), the analytics server may acquire a medical image (e.g., a CT image will be acquired for the patient). In some embodiments, the analytics server may only receive a medical image showing the tumor of the patient without receiving any additional data.

At step 204, the analytics server may execute an artificial intelligence model to predict deformation data for at least one internal structure of the patient, wherein the artificial intelligence model is trained in accordance with a training dataset comprising a set of participants and their corresponding deformation data.

As used herein, deformation data may refer to any data predicted by the AI model that indicates how an internal structure of the patients would deform. In some embodiments, the deformation data may indicate that a segment of an internal structure of a patient will not deform. Therefore, not all the deformation data indicates an actual physical movement of a segment of an internal structure of the patient. Non-limiting examples of deformation data may include any data (e.g., deformation vectors, numbers, and synthetic medical images) that convey how one or more internal structures would move or deform at a given time.

The deformation data may also include a corresponding time window. For instance, the AI model may predict the deformation of one or more segments of one or more internal structures of the patient within a specific time period, such as one day, week, or month.

Using the data received in the step 202 (e.g., the medical image), the AI model may use various techniques to contour the received medical image. Therefore, distinct contours of the patient's internal structures may be segmented. Using the medical image and the planning contours, the AI model may be instantiated for each of the internal structures (e.g., organs) and may be assembled to form a digital twin of the patient. By appropriately actuating the AI model, the analytics server can then generate simulated CBCT images that represent the patient's anatomy at different levels of normal physiological changes and treatment-induced physiological regressions.

The AI model may use a Material Point Method (MPM) to predict the anatomical changes to the internal structures of the patient. As used herein, MPM may refer to a numerical technique used by the AI model to quantitatively simulate the behavior of solids, liquids, gases, and any other continuum material. In the MPM, a given organ may be represented by a few small Lagrangian elements referred to as “material points.” These material points may be surrounded by a background mesh/grid that is used to calculate terms, such as the deformation gradient. Unlike other mesh-based methods (e.g., the finite element method, finite volume method, or finite difference method), the MPM may be categorized as a meshless/mesh-free or continuum-based particle method, examples of which are smoothed particle hydrodynamics. Despite the presence of a background mesh, the MPM may not encounter the drawbacks of mesh-based methods (high deformation tangling, advection errors, etc.) allowing the AI model to predict the deformation data more efficiently.

Using the data received, the AI model may predict deformation data for the patient's one or more internal structures.

At step 206, the analytics server may output the data predicted by the AI model (deformation data). The analytics server may output the deformation data in multiple ways. In one embodiment, the analytics server may output the deformation vectors in a visual form, machine-readable form, and/or human-readable form.

In a first non-limiting example, the AI model to generate a synthetic medical image and display the synthetic medical image on a platform viewable by a clinician and treating physician. In another example, the analytics server may use the AI model to generate a moving or fixed medical image that depicts how the patient's internal structure would move/deform. For instance, a GUI accessed by a medical professional may display a projected 4DCT of the patient that depicts how the patient's internal structures are going to move/deform.

In a second non-limiting example, the AI model may generate machine-readable data indicating how one or more internal structures will deform within a specified time period. The analytics server may then transmit the machine-readable data to another model. That is, the AI model may generate the data needed for DIR optimization. In some embodiments, the results predicted by the AI model may be exported in the form of synthetic CBCT images and may be given as input to the DIR hyper-parameter optimization process, where for given physiological changes, the corresponding accurate DIR hyper-parameters may be predicted.

Using the synthetic medical image (e.g., CBCTs generated from the AI model), the DIR may be trained and optimized for the specific physiological regression. Specific sequences of anatomical changes that include normal and physiological regression may be used and the optimal DIR may be employed.

In some embodiments, a supervised learning process may be instantiated that can ingest the medical image (e.g., patient CT image) and the synthetic medical image generated by the AI model (e.g., synthetic CBCT) as data and the optimal DIR hyper-parameters as a vector of the label. Upon completion, the AI model may predict the optimal DIR hyper-parameter given the image pairs. During the inferencing stage, the optimal DIR registration may then be performed, and the anatomical changes may be computed.

In a third non-limiting example, the analytics server may transmit the deformation data to a plan optimizer computer model. As used herein, the plan optimizer computer model refers to a computer model that uses various algorithmic methods to generate an RTTP for the patient. Using the deformation data, the plan optimizer may revise one or more attributes of the patient's RTTP. This is because contours and segmentations of the deformed PTV and/or OARs may necessitate a change in the patient's RTTP. Accordingly, certain attributes of the RTTP (as originally identified by the plan optimizer) may no longer be within the treatment objectives indicated by the treating physician because the anatomy of the patient is predicted to have changed. Using the new data, the plan optimizer computer model may predict a new/revised RTTP for the patient.

In some embodiments, as a result of the new/revised RTTP, the analytics server may revise one or more attributes/configurations of a radiation therapy machine. For instance, the analytics server may revise an attribute of a multi-leaf collimator (MLC), move the couch, pause the beam, or a combination of any of these examples. Specifically, in conjunction with one or more other software solutions, the analytics server may revise an opening of the MLC, such that radiation dissemination is directed towards the projected location of a PTV (e.g., projected using the AI model). In this way, the analytics server provides a dynamic MLC correction method where the MLC opening can be revised in real-time or near real-time. Effectively, the analytics server may enable the gating of the beam to match the (predicted) deformations of the patient's tumor. Because the analytics server can predict/estimate the tumor location, the analytics server may control one or more attributes of the radiation therapy machine. For instance, the analytics server may control (e.g., review and revise) the MLC opening, timing, and/or dose rate.

In another example, the analytics server may transmit the data predicted via the AI model to a downstream software solution. For instance, using the results of the execution of the AI model can be transmitted to a dose calculation software solution. In another example, the analytics server may transmit the deformation data to a downstream tissue-tracking application.

Many conventional approaches for treatment monitoring do not include an optimal tracking of systematic anatomical changes. Instead, they include a simplistic online dose verification solution. This can lead to scenarios, where the anatomy tracking remains unreliable and so the treatment is not modified. Accordingly, additional medical image (e.g., CT) acquisitions may lead to an under-usage of the treatment systems. In contrast, using the methods and systems discussed herein has the advantage of using AI modeling techniques in a biomechanics-based framework that enables an optimal tracking of the entire patient anatomy for all physiological regression and planning for a treatment re-plan for each patient. This can ensure that clinical centers can always conduct in a highly efficient manner while reliably identifying patients that need treatment adaptation.

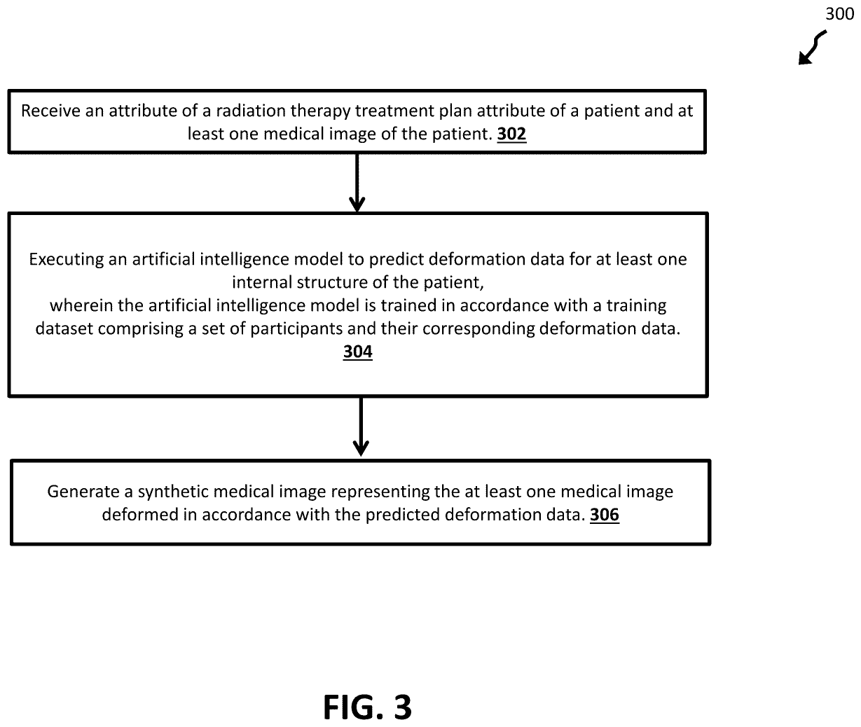

FIG. 3 depicts an example data flow diagram illustrating a method 300 that shows how an AI model can be trained and executed to predict a patient attribute, in accordance with an embodiment. The method 300 may include steps 302-306. However, other embodiments may include additional or alternative steps or may omit one or more steps altogether. The method 300 is described as being executed by a server, such as the analytics server described in FIG. 1. However, one or more steps of method 300 may be executed by any number of computing devices operating in the distributed computing system described in FIG. 1. For instance, one or more computing devices may locally perform part or all of the steps described in FIG. 3.

Using the method 300, the analytics server may generate a synthetic medical image that corresponds to the predicted deformations of one or more anatomical regions of a patient.

At steps 302 and 304, the analytics server may receive a radiation therapy treatment plan attribute of a patient and at least one medical image of the patient and may further execute an artificial intelligence model to predict deformation data for at least one internal structure of the patient, wherein the artificial intelligence model is trained in accordance with a training dataset comprising a set of participants and their corresponding deformation data. The steps 302 and 304 may be similar to the steps 202 and 204. Using the methods and systems discussed herein, the analytics server may identify deformation data for the patient.

At step 306, the analytics server may generate a synthetic medical image representing the at least one medical image deformed in accordance with the predicted deformation data. As discussed herein, the AI model can be trained to generate a synthetic medical image that illustrates the patient's anatomical regions deformed in accordance with the deformation data. Accordingly, the synthetic image may illustrate how the patient's internal structures would (are predicted to) deform.

The synthetic medical image may then be ingested by a downstream software application, such as the plan optimizer computer model and/or any algorithm using the DIR-related data. Using the deformation data, the downstream software application may then revise the RTTP, as discussed herein.

The type of the synthetic image may or may not correspond to the medical image ingested by the AI model. For instance, in some embodiments, the AI model may ingest a CT image of the patient, predict deformation data associated with the patient's internal structures, and generate a synthetic CBCT for the patient.

In some embodiments, the synthetic medical may include one or more deformation vectors. For instance, the analytics server (via the AI model) may generate vectors that indicate how each segment of the patient's internal structures are predicted to move (or not move). Non-limiting examples of the deformation vectors are depicted in FIG. 4. The vector may visually represent the direction and magnitude of how each segment of the patient's internal structure is predicted to deform/move. The analytics server may also display numerical indicators conveying distances and directions of the movement for each segment (e.g., each voxel).

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limited of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
receiving, by a processor, an attribute of a radiation therapy treatment plan for a patient and at least one medical image of the patient;
executing, by the processor, an artificial intelligence model to predict deformation data for at least one internal structure of the patient using the attribute and the at least one medical image, wherein the artificial intelligence model is trained in accordance with a training dataset comprising a set of participants and their corresponding deformation data; and
outputting, by the processor, the predicted deformation data.

2. The method of claim 1, wherein the deformation data corresponds to a movement, volume expansion, or volume shrinkage of the at least one internal structure of the patient.

3. The method of claim 1, wherein the deformation data is a hyperparameter used by a second model configured to predict a movement, volume expansion, or volume shrinkage of the at least one internal structure of the patient.

4. The method of claim 3, further comprising:
transmitting, by the processor, the hyperparameter to the second model.

5. The method of claim 1, further comprising:
transmitting, by the processor, the deformation data to a plan optimizer computer model.

6. The method of claim 1, further comprising:
adjusting, by the processor, at least one attribute of a radiation therapy machine in accordance with the predicted deformation data.

7. The method of claim 1, wherein the deformation data corresponds to one or more deformation vectors.

8. A non-transitory machine-readable storage medium having computer-executable instructions stored thereon that, when executed by one or more processors, cause the one or more processors to perform operations comprising:
receive an attribute of a radiation therapy treatment plan for a patient and at least one medical image of the patient;
execute an artificial intelligence model to predict deformation data for at least one internal structure of the patient using the attribute and the at least one medical image, wherein the artificial intelligence model is trained in accordance with a training dataset comprising a set of participants and their corresponding deformation data; and
output the predicted deformation data.

9. The non-transitory machine-readable storage medium of claim 8, wherein the deformation data corresponds to a movement, volume expansion, or volume shrinkage of the at least one internal structure of the patient.

10. The non-transitory machine-readable storage medium of claim 8, wherein the deformation data is a hyperparameter used by a second model configured to predict a movement, volume expansion, or volume shrinkage of the at least one internal structure of the patient.

11. The non-transitory machine-readable storage medium of claim 10, wherein the instructions further cause the one or more processors to transmit the hyperparameter to the second model.

12. The non-transitory machine-readable storage medium of claim 8, wherein the instructions further cause the one or more processors to transmit the deformation data to a plan optimizer computer model.

13. The non-transitory machine-readable storage medium of claim 8, wherein the instructions further cause the one or more processors to adjust at least one attribute of a radiation therapy machine in accordance with the predicted deformation data.

14. The non-transitory machine-readable storage medium of claim 8, wherein the deformation data corresponds to one or more deformation vectors.

15. A system comprising a processor configured to:
receive an attribute of a radiation therapy treatment plan for a patient and at least one medical image of the patient;
execute an artificial intelligence model to predict deformation data for at least one internal structure of the patient using the attribute and the at least one medical image, wherein the artificial intelligence model is trained in accordance with a training dataset comprising a set of participants and their corresponding deformation data; and
output the predicted deformation data.

16. The system of claim 15, wherein the deformation data corresponds to a movement, volume expansion, or volume shrinkage of the at least one internal structure of the patient.

17. The system of claim 15, wherein the deformation data is a hyperparameter used by a second model configured to predict a movement, volume expansion, or volume shrinkage of the at least one internal structure of the patient.

18. The system of claim 17, wherein the processor is further configured to transmit the hyperparameter to the second model.

19. The system of claim 15, wherein the processor is further configured to transmit the deformation data to a plan optimizer computer model.

20. The system of claim 15, wherein the processor is further configured to adjust at least one attribute of a radiation therapy machine in accordance with the predicted deformation data.
